(19) United States
(12) Patent Application Publication
(10) Pub. No.: US 2005/0119760 A1
(43) Pub. Date: Jun. 2, 2005

(54) Body joint replacement titanium implant comprising one or several base bodies

(76) Inventors: Hans-Georg Neumann, Rostock (DE); Peter Zeggel, Sildemow (DE); Giles Melsom, Bennekom (NL); Egidius Hermsen, Eindhoven (NL)

Correspondence Address: JORDAN AND HAMBURG LLP, 122 EAST 42ND STREET, SUITE 4000, NEW YORK, NY, 10168, US

(21) Appl. No.: 10/504,737

(22) PCT Filed: Feb. 10, 2003
(86) PCT No.: PCT/EP03/01275

(30) Foreign Application Priority Data
Feb. 15, 2002 (DE) 102 06 627.2

(51) Int. Cl. A61F 2/28

(52) U.S. Cl. 623/23.53; 623/23.36; 623/23.57

(57) ABSTRACT

The invention relates to a body joint replacement titanium implant comprising one or several base bodies. In known titanium implants, there is a risk of loosening due to the anchoring of the implants in the bone at points where an optimum bone regeneration is undesirable. The functioning of the joints of the implants can be impaired by coalescing with connective tissue. The novel titanium plants avoid said problems by virtue of the fact that parts (3, 4, 8, 9) of the implant are provided with an anodically (type II) oxidised layer containing oxygen and silicon.

The invention relates to a titanium implant comprising one or several base bodies which anchor in the bone.

In orthopedics, titanium or titanium alloys are used as a material for producing implants because, on the one hand, it is lighter than steel alloys and, on the other hand, it is biocompatible. This allows the implant to coalesce with bone cells for example.

In many implants, however, coalescence is desired only to a certain extent. For example, in order to achieve good anchoring of a cementless hip implant, it is necessary to stimulate growth of bone cells on its proximal part, in order thereby to achieve a good connection between the bone and the implant. Since, as Wolff's law teaches, bone regeneration is at its greatest at the place where the optimal physiological load exists, there is a risk of the area of bone regeneration migrating to the distal part of the implant. Bone growth is stimulated to the greatest extent in this area, and the implant is connected most strongly to the bone there. There is then a danger of bone being destroyed in the proximal area and of the implant coming loose in this area. In the worst case, this can lead to fracturing of the implant. To avoid the danger of loosening of the implant, hip implants in some cases have a specially configured proximal area which is provided with a roughened surface and/or coating of calcium phosphates on which bone growth is promoted or good anchoring of the bone is to be achieved. However, these measures cannot prevent regeneration of bone and therefore anchoring of the implant in the distal area. To counteract loosening of the implant in the proximal area, other implants have polished surfaces in the distal area. A polished surface does delay the formation of bone cells on the metal surface, but it is not able to prevent solid anchoring in the bone.

The object of the present invention is to configure the surface, in that area of a titanium implant where coalescence with body tissue or bone cells is desirable, in such a way that new formation of bone cells on the metal surface is impeded.

According to the invention, the object is achieved by the fact that a part of the titanium implant has a layer which is produced by anodic oxidation (type II) and which contains oxygen and silicon.

The process of anodic oxidation (type II) is described in AMS 2488, issued by SAE International, Warrendale, USA, 1994. In anodic oxidation (type II), no layer of TiO₂ is formed, and instead oxygen and silicon are introduced into the layer near the surface, referred to as the conversion layer, and form an integral component of the material. This layer on the one hand impedes the adherence of bone cells and on the other hand ensures an increased fatigue strength of the titanium material. For a hip implant, this means that, in the anodically (type II) oxidized area, no solid anchoring of the implant to the bone takes place. The load area, which is necessary for the formation of bone, therefore remains restricted to the proximal area of the implant. Loosening of the implant is avoided in this way. However, if coalescence does takes place in the longer term in the distal area, the danger of fracturing of the implant is reduced by means of the increased fatigue strength. The above observations also apply to knee implants.

In titanium implants with movable plug connections, coating of the plug connection impedes coalescence with connective tissue. A further advantage lies in the fact that the sliding properties of the plug connection are improved, since the coating prevents cold welding of the two parts of the plug connection.

Advantageous embodiments of the implant according to the invention are described in the dependent claims.

Examples of the invention are discussed in more detail below with reference to drawings in which:

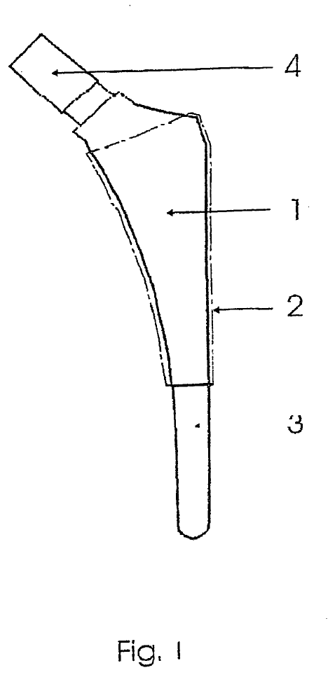

FIG. 1 shows a diagrammatic representation of a hip implant, and

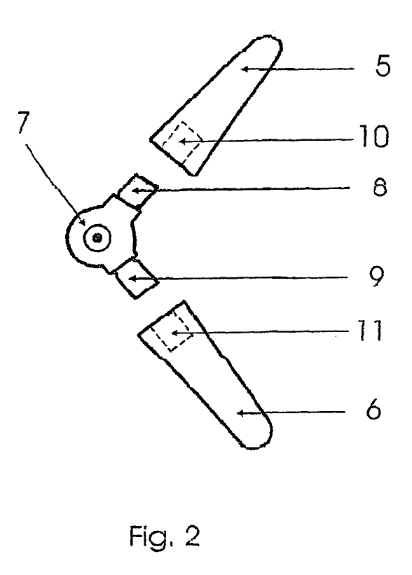

FIG. 2 shows a diagrammatic representation of a finger implant.

The hip implant in FIG. 1 consists of a base body 1 which, in its proximal part, has an area 2 that is intended to be firmly anchored to the bone. This area preferably has a rough surface which can be produced by sandblasting with coarse-grained material or by a titanium coating. To stimulate bone growth, it is advantageous to coat the rough surface with one or more absorbable calcium phosphate layers. In the distal part of the implant, this area is adjoined by an area 3 which is not intended to coalesce with the bone and is therefore anodically (type II) oxidized. In this surface modification, oxygen and silicon are introduced into the titanium surface and there form an integral component of the conversion layer. The area has no TiO₂ layer. If so desired, carbon and nitrogen can also be introduced into the conversion layer.

The conversion layer on the one hand impedes the adherence of bone cells. However, it also leads to greater hardness and thus to a greater fatigue strength of this part and reduces frictional corrosion.

The hip implant also has a cone 4 for a plug-on head. Since, in revision surgery, it is sometimes desirable for the base body of the hip implant to remain in the bone, it is advantageous also for the cone 4 to be provided with an anodically (type II) oxidized layer. It is then easier to remove the plug-on head from the main body. In addition, the greater hardness protects against damage to the cone.

FIG. 2 shows a finger joint implant consisting of the base bodies 5 and 6 which are intended to coalesce with the corresponding bones of the finger. They have a rough surface and are advantageously provided with one or more absorbable calcium phosphate layers. The two base bodies 5 and 6 are connected via a hinge part 7 with cones 8 and 9 which are inserted into anchoring sleeves 10, 11 located in the base bodies 5 and 6. To permit movement of the cones 8 and 9 in the sleeves 10, 11, a movable arrangement is necessary. The mobility must also be guaranteed after implantation of the finger joint implant and must not be impeded by coalescence with connective tissue. The cones 8 and 9 and the anchoring sleeves 10, 11 are therefore also provided with an anodic oxidation layer (type II). In addition to suppressing the adherence of connective tissue, the coating has the advantage that cold welding of the cones to the anchoring sleeves is prevented and good sliding surfaces are formed.

1. A body joint replacement titanium implant comprising at least one base body which armor anchors in the bone, a part of the implant being provided with an anodically (type II) oxidized layer comprising oxygen and silicon.

2. The titanium implant as claimed in claim 1, wherein the layer is selected from the group consisting of carbon and nitrogen.

3. The titanium implant as claimed in claim 1 or 2, comprising an unlayered part devoid of said layer, said unlayered part connecting with a bone, said unlayered part having a rough surface.

4. The titanium implant as claimed in claim 3, wherein the rough surface is coated with absorbable calcium phosphates.

5. The titanium implant as claimed in claim 4, wherein the calcium phosphate comprises several layers.

6. The titanium implant as claimed in claim 1, wherein the implant is a hip implant.

7. The titanium implant as claimed in claim 1, 2 or 6 comprising a cone for a plug-on head provided with the anodically (type II) oxidized layer.

8. The titanium implant as claimed in claim 1 wherein the implant is a knee implant.

9. The implant as claimed in claim 1, wherein the implant is a finger joint comprising two base bodies which are connected to one another via a hinge, said hinge being provided with an anodically (type II) oxidized layer.

10. The implant as claimed in claim 9, wherein the finger implant comprises base bodies having anchoring sleeves, said hinge comprising a hinge part separate from said base bodies, said hinge part having cones, the cones of the hinge part and the anchoring in the base bodies being provided with the anodically (type II) oxidized layers.
